(19) United States
(12) Patent Application Publication
(10) Pub. No.: US 2008/0274198 A1
(43) Pub. Date: Nov. 6, 2008

(54) USE OF MULTILAYERED PIGMENTS IN THE FOOD AND PHARMACEUTICALS SECTOR

(76) Inventor: Ralf SCHWEINFURTH, Darmstadt (DE)

Correspondence Address: MILLEN, WHITE, ZELANO & BRANIGAN, P.C., 2200 CLARENDON BLVD., SUITE 1400, ARLINGTON, VA, 22201, US

(21) Appl. No.: 12/170,095

(22) Filed: Jul. 9, 2008

Related U.S. Application Data
(63) Continuation of application No. 10/503,241, filed on Aug. 2, 2004, now abandoned.

(30) Foreign Application Priority Data
Feb. 1, 2002 (DE) 102 04 336.1

(51) Int. Cl. A61K 9/14; A61K 8/02; A23L 1/275

(52) U.S. Cl. 424/489; 426/540

(57) ABSTRACT

The present invention relates to the use of multilayered pigments based on platelet-shaped substrates for colouring food and pharmaceutical products.

The present invention relates to the use of multilayered pigments based on platelet-shaped substrates for colouring food and pharmaceutical products.

Besides the functional applications, multilayered pigments are also increasingly being employed for improving the appearance of products, for example in cosmetics, since attractive colours and effects cause pleasant subjective sensations in observers and consumers. It should also be possible to employ multilayered pigments in the food and pharmaceuticals sector for improving the colour effect or for colouring, so long as they have been prepared in accordance with the corresponding, strict purity and quality regulations.

The use of pearlescent or interference pigments for colouring products from the food and pharmaceuticals sector is disclosed in DE 198 31 869. However, these products have the disadvantage that the original colour of the product to be coloured is covered to a greater or lesser extent. It is, however, frequently desired for the original product colour to be retained and merely refined through an additional colour lustre effect. As a consequence, the dye content in the product to be coloured can also be reduced before colouring with the multilayered pigments.

The object of the present invention is therefore to widen the range of hues already known in the colouring of food and pharmaceutical products, causing the products to experience an additional sensorily perceptible improvement. The optical upgrading is particularly valuable for pharmaceutical products since clearer differentiation of differently coloured coated and uncoated tablets, etc., is facilitated.

Surprisingly, it has now been found that multilayered pigments based on platelet-shaped substrates coated with alternating layers of metal oxides are highly suitable for improving the appearance of food and pharmaceutical products. Particularly preferred metal oxides are TiO₂, Fe₂O₃ and Fe₃O₄. In particular, the combination of multilayered pigments having TiO₂ and/or Fe₃O₄ layers with natural or nature-identical dyes, coloured pigments or colouring fruit and plant extracts give the food product an interesting new hue. The invention thus relates to the use of multilayered pigments based on platelet-shaped substrates for colouring food and pharmaceutical products.

Compared with the products from DE 198 31 869, the products which have been coloured with a multilayered pigment have significantly greater tinting strength and transparency, higher lustre and exhibit a colour flop between two or more colours.

The coloured foods and pharmaceutical products are distinguished by a multicoloured effect which causes pleasant subjective sensations in observers and consumers. This optical effect is not possible with the colorants currently approved in the food sector. In contrast to coloured pigments which are approved in the food sector, for example vegetable black E153, the multilayered pigments based on platelet-shaped substrates can be dispersed very easily into the medium to be pigmented. Furthermore, the products coloured in this way are distinguished by increased light and moisture protection. In particular, vitamin preparations have a longer shelf life. In the case of colouring of tablets, delayed release of active ingredients has been observed in many cases.

It has been found that novel interesting hues and at the same time novel properties can be imparted on the foods or pharmaceutical products even with very small amounts of multilayered pigments. Excellent results are achieved on colouring of the product with only from 0.0025 to 75.0% by weight, preferably from 0.0025 to 50% by weight, in particular from 0.05 to 25% by weight, of multilayered pigment, based on the product.

In the case where the pigment is admixed directly with the food or pharmaceutical product during production, the amount of multilayered pigment used is preferably from 0.005 to 4% by weight. In the case of surface treatment of foods or tablets, the use range is from 0.02 to 15.0% by weight, preferably from 0.5 to 6.0% by weight, based on the pigment or coating solution.

Suitable multilayered pigments are pigments based on multicoated substrates which are distinguished by the fact that they have alternating high- and low-refractive-index metal-oxide layers.

Particularly suitable are multilayered pigments based on multicoated platelet-shaped substrates which comprise at least one layer sequence (A) (B) (A), where
(A) is a high-refractive-index coating consisting of titanium dioxide and/or iron oxide, and
(B) is a low-refractive-index coating having a refractive index of n≦1.8.

Suitable base substrates for the multilayered pigments according to the invention are transparent or semi-transparent platelet-shaped substrates. Preferred substrates are phyllosilicates. Particularly suitable are natural and/or synthetic mica, talc, kaolin, platelet-shaped iron oxides or aluminium oxides, glass, SiO₂, TiO₂, platelet-shaped mixed oxides, such as, for example, FeTiO₃, Fe₂TiO₅, or other comparable materials, depending on the particular legal permissibility for use in foods or pharmaceutical products.

The size of the base substrates is not crucial per se and can be matched to the particular application. In general, the platelet-shaped substrates have a thickness of between 0.005 and 10 μm, in particular between 0.05 and 5 μm. The extension in the two other directions is usually between 1 and 500 μm, preferably between 2 and 200 μm, and in particular between 5 and 60 μm.

The thickness of the individual layers (A) and (B) of high or low refractive index on the base substrate is essential for the optical properties of the pigment. For the multilayered pigment having an intense lustre effect, the thickness of the individual layers must be set precisely with respect to one another.

The thickness of layer (A) is 10- 500 nm, preferably 20- 400 nm, in particular 30-350 nm. The thickness of layer (B) is 10-500 nm, preferably 20-400 nm, in particular 30-350 nm.

The pigments can comprise a plurality of identical or different combinations of layer packages, but the substrate is preferably coated with only one layer package (A) (B) (A). In order to intensify the tinting strength, the pigment according to the invention can comprise up to 4 layer packages, but where the thickness of all layers on the substrate should not exceed 3 μm. An odd number of layers is preferably applied to the platelet-shaped substrate, each having a high-refractive-index layer in the innermost and outermost layer. Particular preference is given to a structure of three optical interference layers in the sequence (A) (B) (A). Suitable high-refractive-index layers are preferably TiO₂, Fe₂O₃ and/or Fe₃O₄. The TiO₂ here can be in the rutile or anatase modification.

Colourless, low-refractive-index materials which are suitable for coating (B) and are suitable in accordance with legal approvals for use in foods or pharmaceutical products are preferably metal oxides or the corresponding oxide hydrates, such as, for example, SiO₂, Al₂O₃, AlO(OH), B₂O₃, MgF₂, MgSiO₃ or a mixture of the said metal oxides. Layer (B) is, in particular, an SiO₂ layer.

The known multilayered pigments can easily be prepared by the production of a plurality of high- and low-refractive-index interference layers having a precisely defined thickness and a smooth surface on the finely divided, platelet-shaped substrates.

The metal-oxide layers are preferably applied by wet-chemical methods, it being possible to use the wet-chemical-coating methods developed for the preparation of pearlescent pigments. Methods of this type are described, for example, in DE 14 67 468, DE 19 59 988, DE 20 09 566, DE 22 14 545, DE 22 15 191, DE 22 44 298, DE 23 13 331, DE 25 22 572, DE 31 37 808, DE 31 37 809, DE 31 51 343, DE 31 51 354, DE 31 51 355, DE 32 11 602, DE 32 35 017 and also in further patent documents and other publications known to the person skilled in the art.

The coating can furthermore also be carried out in a fluidised-bed reactor by gas-phase coating, it being possible to use correspondingly, for example, the processes proposed in EP 0 045 851 and EP 0 106 235 for the preparation of pearlescent pigments.

The hue of the multilayered pigments can be varied in very broad limits through a different choice of the coating amounts or the layer thicknesses resulting therefrom. Fine tuning for a certain hue can be achieved beyond the pure choice of amounts by approaching the desired colour with visual or measurement technology control.

Particularly preferred multilayered pigments have the following layer structure:

substrate + TiO₂ + SiO₂ + TiO₂
substrate + TiO₂ + SiO₂ + Fe₂O₃
substrate + TiO₂ + SiO₂ + Fe₃O₄
substrate + TiO₂ + Al₂O₃ + TiO₂
substrate + TiO₂ + Al₂O₃ + Fe₂O₃
substrate + TiO₂ + Al₂O₃ + Fe₂O₃
substrate + Fe₂O₃ + SiO₂ + TiO₂
substrate + Fe₃O₄ + SiO₂ + TiO₂
substrate + Fe₂O₃ + Al₂O₃ + TiO₂
substrate + Fe₃O₄ + Al₂O₃ + TiO₂

In particular, the above-mentioned multilayered pigments are based on mica platelets, furthermore on glass, SiO₂ or Al₂O₃ platelets.

The multilayered pigments used are preferably magnetite- and TiO₂-coated natural or synthetic mica platelets.

The admixing of pearlescent pigments, coated or uncoated TiO₂, and/or SiO₂ platelets, natural or nature-identical dyes, organic or inorganic coloured pigments or colouring natural fruit and plant extracts which are approved for the food sector enables the colour effect of the multilayered pigments in the product to be influenced and at the same time enables novel iridescent colour effects to be achieved.

Besides the multilayered pigments, all natural or nature-identical dyes which are known to the person skilled in the art can be admixed as further colouring component. In particular, mention should be made here of E 101, E 104, E 110, E 124, E 131, E 132, E 140, E 141, E 151 and E 160a. Furthermore, it is also possible to admix other coloured pigments with the platelet-shaped pearlescent pigments, such as, for example, E 171, E 172 and E 153.

The proportion of dyes, based on the product, is in the range from 0.5 to 25% by weight. The dyes employed can likewise be fruit and plant extracts, such as, for example, carrot juice, beetroot juice, elderberry juice, hibiscus juice, paprika extract and aronia extract.

The respective colour effect of the pigments is reinforced through the combination of the multilayered pigments with other pearlescent pigments, such as gold, silver or interference pigments. This synergism considerably broadens the range of possible colours of the products to be pigmented without the need additionally to employ other natural or nature-identical dyes.

The multilayered pigments which are approved for the food sector are commercially available, for example under the trade name Candurin® ML from Merck KGaA.

The total concentration of all pigments in the product to be pigmented should likewise not exceed 12% by weight, based on the product. It is generally dependent on the specific application.

The pharmaceutical and food products are coloured by adding the multilayered pigment to the product to be coloured, alone or in combination with further pigments or colorants, directly or in the presence of water and/or an organic solvent in the desired mixing ratios, at the same time or successively, during or after production thereof. Complex grinding and dispersal of the pigments is not necessary.

On incorporation into the product matrix, for example fruit gum, beverages, etc., itself, the amount of multilayered pigments used is preferably 0.0025-10% by weight, in particular 0.05-3% by weight. In the case of surface colouring of foods and pharmaceutical products, for example tablets, confectionery, etc., the amount used in the pigment or coating solution used is in the range 0.01-30% by weight, in particular 0.1-15% by weight. On use of the multilayered pigments in pulverulent products, the amount used is in the range 0.05-50% by weight, in particular 2-10% by weight.

The coating solutions preferably comprise water or organic solvents, preferably ethanol or isopropanol. The film former employed in the coating solutions is preferably a cellulose derivative, such as, for example, hydroxypropylmethylcellulose. Particular preference is given to application solutions comprising cellulose derivatives which, instead of water, comprise 5-80% by weight of a suitable organic solvent.

Compared with aqueous coating solutions, the alcoholic or alcoholic-aqueous, cellulose-containing application solutions have significant applicational advantages:
use of cooler drying air during the spray application
colouring of heat-sensitive products, such as, for example, chocolate figures, liquorice, chocolate coated tablets, etc., with multilayered pigments is readily possible.

Further products that may be mentioned which are suitable for colouring are, in particular, coatings on all types of foods, in particular pigmented sugar and shellac coatings (alcoholic and aqueous), coatings with oils, fats, starches and waxes, with gum arabic, with cellulose derivatives (for example HPMC=hydroxypropylmethylcellulose), with starch and albumen derivatives, carrageenan and other substances known to the person skilled in the art which are suitable for coating, the incorporation or application to sugar products, cake decorations, compresses, coated tablets, chewing gum, gum products, fondant products, marzipan products, filling compositions, cocoa and fat glazes, chocolate and chocolate-containing products, ice cream, cereals, snack products, coating compositions, cake glazes, scattered sugar decorations, nonpareils, jelly and gelatine products, sweets, liquorice, icing, candyfloss, fat, sugar and cream compositions, blancmange, desserts, flan glaze, cold fruit soups, foods in powder form, beverages, with and without stabilising additives, such as, for example, carboxymethylcellulose, acidified and unacidified milk products, such as, for example, quark, yoghurt, cheese, cheese rinds, sausage casings, etc.

A further major area of application is in the pharmaceutical and OTC sector for colouring tablets, hard and soft gelatine capsules, capsettes, coated tablets, ointments, cough mixture, products in liquid and pulverulent form, etc. In combination with conventional coatings, such as polymethacrylates and cellulose types, for example HPMC, the multilayered pigments can be employed in a variety of ways for colouring.

In the case of coated food and pharmaceutical products, the combination of the multilayered pigments with aroma substances (powder or liquid aromas), acids and/or with sweeteners, such as, for example, aspartame, is possible in order additionally to emphasise the optical effect in taste terms.

The invention thus relates to all formulations from the food and pharmaceuticals sector comprising one or more multilayered pigments, alone or in combination with further pigments/pigment mixtures or dyes (natural or nature-identical) as colorants.

The following examples are intended to explain the invention, but without limiting it.

### EXAMPLES

### Example 1

### Fruit Gum Products

The fruit gum products are produced in the usual way. The use of highly transparent gel formers improves the desired colour effect.

The multilayered pigments are pre-suspended in water, aromas and/or sugar. This prevents the formation of undesired and irreversible pigment agglomerates.

### 1. Formulation Example

| Component | Percentage | Source of supply |
|---|---|---|
| Water | 10.6945% | |
| Sugar (sucrose) | 31.45% | Südzucker |
| Glucose syrup | 31.45% | Cerestar, Krefeld |
| Candurin ® Blue ML** | 0.285% (0.3% based on | Merck KGaA |
| | the casting composition) | |
| Citric acid 1:1 dil. | 2.51% | Merck KGaA, |
| Gelatine (260 Bloom) | 7.86% | DGF, Eberbach |
| Water | 15.748% (For dissolving | |
| | the gelatine) | |
| Dye E129 | 0.0025% | BASF |
| Aroma | Depending on the | |
| | desired taste direction | |

**Multilayered pigment based on mica coated with TiO₂ and SiO₂

### 2. Formulation Example

| Component | Percentage | Source of supply |
|---|---|---|
| Water | 10.598% | |
| Sugar (sucrose) | 31.45% | Südzucker |
| Glucose syrup | 31.45% | Cerestar, Krefeld |
| Candurin ® Yellow ML** | 0.38% (0.4% based on | Merck KGaA |
| | the casting composition) | |
| Citric acid 1:1 dil. | 2.51% | Merck KGaA |
| Gelatine (260 Bloom) | 7.86% | DGF, Eberbach |
| Water | 15.748% (for dissolving | |
| | the gelatine) | |
| Dye E133 | 0.0022% | BASF |
| Dye E102 | 0.0018% | BASF |
| Aroma | Depending on the | |
| | desired taste direction | |

**Multilayered pigment based on mica coated with TiO₂ and SiO₂

### Preparation:

Firstly, the gelatine is softened or pre-swollen with twice the amount of water at 60° C. Sugar and water are heated to 100° C. The glucose syrup is then added. The mixture is heated further to 120° C. and then allowed to cool to 85° C. The multilayered pigment, citric acid, aroma and gelatine solution are stirred in. The degassed casting composition is transferred using the casting funnel either into greased moulds or into negative moulds stamped in starch powder. The product is allowed to cool for about 10-16 hours. finally, the fruit gum products are removed from the moulds (the powder is removed in the case of starch powder) and treated with a suitable release agent.

The multilayered pigments can be added to the production batch at any desired time owing to their good heat and pH stability.

### Example 2

### Sugar-Coated Products

### 1st Example

Product to be coloured: green, sugar-coated almonds

Composition of the application spray suspension:

| Component | Percentage | Source of supply |
|---|---|---|
| Candurin ® Blue ML | 3% | Merck KGaA, |
| Sepifilm 050 | 5% | Seppic |
| Water | 92% | |

Application rate: depending on the desired colour intensity about 1-4% of spray suspension/kg of product. Temperature of the drying air: 35-45° C.

### 2nd Example

Product to be coloured: brown, sugar-coated hazelnuts with chocolate

Composition of the application spray suspension:

| Component | Percentage | Source of supply |
|---|---|---|
| Candurin ® Gold ML** | 5% | Merck KGaA |
| Sepifilm 050 | 5% | Seppic |
| Water | 50% | |
| Ethanol | 40% | Merck KGaA |

**Multilayered pigment based on mica coated with Fe₂O₃, SiO₂ and TiO₂

Application rate: depending on the desired colour intensity about 2-6% of spray suspension/kg of product. Temperature of the drying air: 28-35° C.

### 3rd Example

Product to be coloured: peanuts coated with dark chocolate

Composition of the application spray suspension:

| Component | Percentage | Source of supply |
|---|---|---|
| Candurin ® Red ML** | 2% | Merck KGaA |
| Sepifilm 050 | 5% | Seppic |
| Water | 33% | |
| Ethanol | 60% | Merck KGaA |

**Multilayered pigment based on mica coated with TiO₂ and SiO₂

Application rate: depending on the desired colour intensity about 1-4% of spray suspension/kg of product. Temperature of the drying air: 26-28° C.

### 4th Example

Product to be coloured: red-coated chewing-gum balls

Composition of the application spray suspension:

| Component | Percentage | Source of supply |
|---|---|---|
| Candurin ® Yellow ML | 3% | Merck KGaA |
| Sepifilm 050 | 5% | Seppic |
| Water | 92% | |

Application rate: depending on the desired colour intensity 1-4% of spray suspension/kg of product

Temperature of the drying air: 35-45° C.

Preparation of the spray suspension:

The multilayered pigments are stirred into or suspended in water. The film former selected is then scattered in slowly with continuous stirring.

Depending on the heat resistance of the products to be coloured, ethanol can now also be added.

Due to the increasing viscosity of the suspension, the stirrer speed has to be matched accordingly. Depending on the composition of the film former, it is completely dissolved immediately in 20-60 minutes. The suspension is then sprayed onto the products. Corresponding drying air is supplied continuously during this operation. As desired, sweeteners or aromas can also be added to the spray suspension.

The application can be carried out in conventional coating reactors, coating apparatuses or in closed film coaters. Suitable film formers are all materials which fix the pigments on the product surface in a highly transparent film (cellulose types, carrageenans, shellac, etc.).

The spray application is generally carried out using a 2-component spray device (airborne).

### Example 3

### Chocolate Articles

### 1st Example

Colouring of hollow chocolate figures (made from dark, white and/or milk chocolate)

Composition of the application spray suspension:

| Component | Percentage | Source of supply |
|---|---|---|
| Candurin ® Blue ML | 3% | Merck KGaA |
| Capol 425 | 19.4% | Kaul GmbH |
| Ethanol | 77.6% | Merck KGaA |

### 2nd Example

Colouring of foam articles covered with chocolate

Composition of the application spray suspension:

| Component | Percentage | Source of supply |
|---|---|---|
| Candurin ® Gold | 5% | Merck KGaA |
| Capol 425 | 19% | Kaul GmbH |
| Ethanol | 76% | Merck KGaA |

### 3rd Example

| Component | Percentage | Source of supply |
|---|---|---|
| Candurin ® Green ML** | 3% | Merck KGaA |
| Sepifilm 050 | 5% | Seppic |
| Ethanol | 50% | Merck KGaA |
| Water | 42% | |

Multilayered pigment based on mica coated with TiO₂ and SiO₂

The spray application can be carried out by means of a spray gun (air-borne) or a conventional airbrush. The pigment application rate is dependent on the desired colour effect.

The use of a high ethanol content in particular enables the colouring of heat-sensitive foods of this type, such as, for example, chocolate products. As a consequence, the processing temperature during the colouring operation can be kept low. The product quality is thus not impaired.

### Example 4

### Beverages

| | Blue beverage | Dark-red beverage | |
|---|---|---|---|
| | with red | with golden | Source of |
| Ingredients | lustre effect | lustre effect | supply |
| Candurin ® Red ML | 0.04% | Gold 0.05% | Merck |
| | | | KGaA |
| Sucrose | 5% | 5% | — |
| Glucose syrup | 7% | 7% | — |
| Citric acid | 0.15% | 0.15% | Merck |
| | | | KGaA |
| Gelcarin (E407) | 0.2% | 0.2% | FMC |
| Type GP-379NF | | | |
| Kelcogel ® (E418) | 0.0075% | 0.0075% | Kelco |
| Aroma | as required | as required | — |
| Additional dye | E131* | Provinol | *BASF |
| | 0.0015% | red-wine | **Seppic |
| | | extract** | Co. |
| | | 0.04% | |
| Water | 87.601% | 87.6025% | — |

pH > 3.5

### Preparation:

(1) Initially introduce water
(2) Add Candurin® (pre-suspended in water)
(3) Add sucrose and glucose syrup
(4) Add thickeners (Gelcarin and Kelcarin®)
(5) Mix vigorously
(6) Heat to 95-100° C. (1-2 minutes) while stirring continuously
(7) Cool to about 40° C. with continuous stirring
(8) Add the citric acid, additional dyes and aromas during the cooling phase
(9) In order to avoid gel formation, the cooled batch should be stirred for a further 30-45 minutes.

If desired, the same results can also be achieved without the addition of corresponding thickeners. Due to the high density of the pigments, they settle relatively quickly. The effect can then be restored by brief shaking.

### Example 5

### Transparent Desserts (For Example Jelly)

| | | E | Source of | |
|---|---|---|---|---|
| Component | (%) | number(s) | supply | |
| Sucrose | 15% | — | — | A |
| Citric acid | 0.38% | E330 | Merck KGaA | C |
| Calcium lactate | 0.05% | E327 | Merck KGaA | A |
| Tripotassium | 0.05% | E340iii | Merck KGaA | A |
| phosphate | | | | |
| Tetrasodium | 0.15% | E450iii | Merck KGaA | A |
| diphosphate | | | | |
| Genugel LC 4N* | 0.6% | E407 + | Kelco | A |
| (Mixture of | | E410 | | |
| carrageenan and | | | | |
| carob seed flour) | | | | |
| Kelcogel (gellan gum) | 0.06% | E418 | Kelco | A |
| Candurin ® Blue ML | 0.04% | E171 + | Merck KGaA | B |
| | | E555 | | |
| Additional dye | 0.0015% | E131 | BASF | C |
| Aroma | as | — | — | C |
| | required | | | |
| Water | 83.6685% | — | — | — |

### Preparation:

(1) Initially introduce water
(2) Add the pulverulent ingredients (A)
(3) Mix well and commence the warming
(4) Add Candurin® (pre-suspended in water) (B)
(5) Heat to 95-100° C. (1-2 minutes), stirring continuously
(6) Cooling phase
(7) Add the citric acid, additional dyes and aromas during the cooling phase (C)
(8) Cool to 40-45° C., stirring continuously
(9) Transfer composition into containers provided

### Example 6

### Tablets

### Preparation:

Tablets are coloured by means of the so-called film coating process. In this, aqueous application solutions (systems comprising film formers, plasticisers, etc.) are sprayed continuously onto the rotating tablets in so-called coaters.

### 1st Example

Product to be coloured: white tablets

Composition of the coating solution:

| Component | Percentage | Source of supply |
|---|---|---|
| Candurin ® Gold ML | 5% | Merck KGaA |
| Sepifilm 050 | 5% | Seppic |
| Water | 90% | |

Application rate: 15-20 g/kg of product

### 2nd Example

Product to be coloured: white tablets

Composition of the coating solution:

| Component | Percentage | Source of supply |
|---|---|---|
| Candurin ® Yellow ML | 3% | Merck KGaA |
| Lustre Clear ® | 2.5% | FMC |
| Water | 94.5% | |

Application rate: 5-10 g/kg of product

### 3rd Example

Product to be coloured: white tablets

Composition of the coating solution:

| Component | Percentage | Source of supply |
|---|---|---|
| Candurin ® Blue ML | 5% | Merck KGaA |
| Opagloss II | 6% | Colorcon |
| Water | 89% | |

Application rate: 5-10 g/kg of product

### 4th Example

Product to be coloured: white tablets

Composition of the coating solution:

| Component | Percentage | Source of supply |
|---|---|---|
| Variochrom Magic | 5% | BASF |
| Purple** | | |
| Sepifilm 050 | 5% | Seppic |
| Water | 90% | |

**Muitilayered pigment based on iron oxide coated with SiO₂ and iron oxide

Application rate: 20-25 g/kg of product

The amount of application solution here depends both on the desired colour effect and on the polymer application required.

1. (canceled)

2. A food or pharmaceutical product according to claim 12, wherein the platelet-shaped substrate is a mica, talc, kaolin, aluminium, Al₂O₃, Fe₂O₃, TiO₂, glass or SiO₂ platelet.

3. A food or pharmaceutical product according to claim 12, wherein the multilayered pigment has alternating high- and low-refractive-index metal-oxide layers.

4. A food or pharmaceutical product according to one of claim 12, wherein the multilayered pigment based on multicoated platelet-shaped substrates comprises at least one layer sequence (A) (B), where
(A) is a high-refractive-index coating consisting of titanium dioxide and/or iron oxide, and
(B) is a colorless coating having a refractive index of n≦1.8.

5. A food or pharmaceutical product according to claim 3, wherein the high-refractive-index layer is TiO₂, Fe₂O₃ and/or Fe₃O₄.

6. A food or pharmaceutical product according to claim 3, wherein the low-refractive-index layer is SiO₂, Al₂O₃, AlO(OH), B₂O₃, MgF₂, MgSiO₃ or a mixture of the said metal oxides.

7. A food or pharmaceutical product according to claim 3, wherein the multilayered pigment has the following layer structure:
substrate + TiO₂ + SiO₂ + TiO₂
substrate + TiO₂ + SiO₂ + Fe₂O₃
substrate + TiO₂ + SiO₂ + Fe₃O₄
substrate + TiO₂ + Al₂O₃ + TiO₂
substrate + TiO₂ + Al₂O₃ + Fe₂O₃
substrate + TiO₂ + Al₂O₃ + Fe₂O₃
substrate + Fe₂O₃ + SiO₂ + TiO₂
substrate + Fe₃O₄ + SiO₂ + TiO₂
substrate + Fe₂O₃ + Al₂O₃ + TiO₂
substrate + Fe₃O₄ + Al₂O₃ + TiO₂

8. A food or pharmaceutical product according to claim 12, wherein the proportion of multilayered pigment in the food or pharmaceutical product is from 0.005 to 15% by weight.

9. A food or pharmaceutical product according to claim 12, wherein the multilayered pigment is employed in combination with one or more pearlescent pigments, coated or uncoated TiO₂ platelets, SiO₂ platelets, natural or nature-identical dyes, coloured pigments or natural colouring plant or fruit extracts.

10. The food or pharmaceutical product according to claim 12, having a coating of cellulose derivatives, shellac, oils, waxes, gum arabic, cellulose products, polymethacrylates, starches, albumens, or icing comprising the multilayered pigment and, optionally, further pigments and/or colorants.

11. A process for the production of a food and pharmaceutical products colored with multilayered pigments, comprising adding the multilayered pigment to the product to be colored, alone or in combination with further pigments or colorants, directly or in the presence of water and/or an organic solvent in the desired mixing ratios, at the same time or successively, during or after production of the food or pharmaceutical product.

12. A food or pharmaceutical product comprising a colorant which is at least one multilayered pigment based on a platelet-shaped substrate, and a food or pharmaceutical.
